(19) United States
(12) Patent Application Publication
(10) Pub. No.: US 2026/0109932 A1
(43) Pub. Date: Apr. 23, 2026

(54) CONTINUOUS SEPARATION APPARATUS AND METHOD FOR MARINE MICROORGANISMS UNDER HIGH-PRESSURE CONDITIONS

(71) Applicant: GUANGDONG LABORATORY OF SOUTHERN OCEAN SCIENCE AND ENGINEERING (GUANGZHOU), Guangdong (CN)

(72) Inventors: Jingchun FENG, Guangdong (CN); Si ZHANG, Guangdong (CN); Yongji HUANG, Guangdong (CN); Yingli ZHOU, Guangdong (CN); Zhifeng YANG, Guangdong (CN)

(73) Assignee: GUANGDONG LABORATORY OF SOUTHERN OCEAN SCIENCE AND ENGINEERING (GUANGZHOU), Guangdong (CN)

(21) Appl. No.: 19/365,226

(22) Filed: Oct. 22, 2025

(30) Foreign Application Priority Data
Oct. 22, 2024 (CN) 202411477969.8

(51) Int. Cl. C12M 1/34; C12M 1/00; C12M 1/26

(52) U.S. Cl. CPC C12M 41/40; C12M 29/06; C12M 33/14; C12M 41/44; C12M 47/02

(57) ABSTRACT

The present invention discloses a continuous separation apparatus and method for marine microorganisms under high-pressure conditions. A microorganism enrichment unit is provided for microbial enrichment; a multi-sequence nutrient solution supply unit is used to supply a nutrient solution required for cultivation; a gas injection unit is used to supply culture gas or inert gas required for cultivation; and a pressure within the microorganism enrichment unit is enabled to be consistent with pressure of an in situ marine where the microorganisms is located; after a period of enrichment culture, a microbial liquid is pumped into a multi-stage separation unit to achieve multi-stage separation of different-size cells of the microbial liquid, thereby obtaining target microbial cells; and finally, the target microbial cells are transferred to a monitoring and cultivation unit for cultivation and stabilization, and an external multi-omics unit is used to perform cell characteristic detection using an external multi-omics unit.

### CROSS-REFERENCE TO RELATED APPLICATION

This application claims the priority benefit of China Application No. 202411477969.8, filed on Oct. 22, 2024. The entirety of the above-mentioned patent application is hereby incorporated by reference herein and made a part of this specification.

### TECHNICAL FIELD

The present invention relates to the technical field of online separation of marine microorganisms, and more specifically, to a continuous separation apparatus and method for marine microorganisms under high-pressure conditions.

### BACKGROUND

Marine microorganisms are simple micro-sized organisms that grow and reproduce in marine environments, and exist as single cells or multicellular forms with relatively simple individual structures, or even have no cellular structures. The ocean, which serves as the habitat for marine microorganisms, is the largest and oldest habitat on the earth. It covers 71% of the earth's surface and has an average depth of 3.8 km. Due to the uneven distribution of land and sea on the earth, the marine habitat is actually composed of many distinct macrohabitats, and different microbial populations thrive under different habitat conditions.

Currently, enrichment and separation experiments related to microorganisms have been conducted under both high pressure and atmospheric pressure conditions. Marine microorganisms are regulated under pressure factors. Through enrichment and separation under atmospheric pressure conditions, only some pressure-tolerant microorganisms can be obtained, but original indigenous microorganisms from the deep sea cannot be obtained. However, microorganisms subjected to enrichment culture under high-pressure conditions are generally separated by streaking or extreme dilution. In addition, if it is necessary to detect microbial characteristics such as gene transcription, environmental pressure needs to be changed, resulting in significant deviations between the obtained results and the actual environment.

### SUMMARY

To overcome a disadvantage that separation and detection of different-sized microorganisms only after pressure release lead to inaccurate detection results during the existing high-pressure enrichment culture process for marine microorganisms, the present invention provides a continuous separation apparatus and method for marine microorganisms under high-pressure conditions. The present invention enables characteristic detection after continuous separation of different-size marine microorganisms under high-pressure conditions. In addition, the present invention features simple operation, improved separation efficiency, accurate detection results, and good conformity to an actual environment.

To solve the above technical problems, the technical solutions of the present invention are as follows:

The present invention discloses a continuous separation apparatus for marine microorganisms under high-pressure conditions, and the continuous separation apparatus for marine microorganisms under high-pressure conditions includes: a microorganism enrichment unit, a gas injection unit, a multi-sequence nutrient solution supply unit, a multi-stage separation unit, a monitoring and cultivation unit, and a central control unit;
a gas inlet end of the microorganism enrichment unit is connected to a gas outlet end of the gas injection unit, and a liquid inlet end of the microorganism enrichment unit is connected to a liquid outlet end of the multi-sequence nutrient solution supply unit;
an input end of the multi-stage separation unit is connected to a liquid outlet end of the microorganism enrichment unit, and an output end of the multi-stage separation unit is connected to an input end of the monitoring and cultivation unit; and
data output ends of the microorganism enrichment unit and the monitoring and cultivation unit are connected to a data input end of the central control unit; and control ends of the microorganism enrichment unit, the gas injection unit, the multi-sequence nutrient solution supply unit, the multi-stage separation unit, and the monitoring and cultivation unit are connected to a control end of the central control unit.

In the present invention, a microorganism enrichment unit is provided for microbial enrichment; a multi-sequence nutrient solution supply unit is used to supply a nutrient solution required for cultivation for the microorganism enrichment unit; a gas injection unit is used to supply culture gas or inert gas required for cultivation microorganism enrichment unit; and a pressure within the microorganism enrichment unit is enabled to be consistent with pressure of an in situ marine where the microorganisms is located, thereby guaranteeing the effectiveness of enrichment culture; after a period of enrichment culture, a microbial liquid is pumped into a multi-stage separation unit to achieve multi-stage separation of different-size cells of the microbial liquid, thereby obtaining target microbial cells; and finally, the target microbial cells are transferred to a monitoring and cultivation unit for cultivation and stabilization, and an external multi-omics unit is used to perform cell characteristic detection using an external multi-omics unit.

Preferably, the multi-stage separation unit includes a first filter, a microorganism separator, a second filter, a first separation filter, a second separation filter, and a multi-channel single-cell sorter;
the first filter, the microorganism separator, the second filter, and the first separation filter are connected in sequence; a first output end of the first separation filter is connected to a first input end of the multi-channel single-cell sorter, a second output end of the first separation filter is connected to an input end of the second separation filter, a first output end of the second separation filter is connected to a second input end of the multi-channel single-cell sorter, and a second output end of the second separation filter is connected to a third input end of the multi-channel single-cell sorter; and
an input end of the first filter serves as the input end of the multi-stage separation unit and is connected to the liquid outlet end of the microorganism enrichment unit; the first output end, second output end, and third output end of the multi-channel single-cell sorter all serve as the output end of the multi-stage separation unit and are connected to the input end of the monitoring and cultivation unit.

The microbial liquid is pumped into the first filter to filter out large particulate impurities, the microbial liquid subjected to primary filtration is obtained and introduced into a microorganism separator for dilution and separation, so as to obtain a separated diluted microbial liquid; the separated diluted microbial liquid is introduced into a second filter to filter out sediments, so as to obtain the microbial liquid subjected to secondary filtration; a filtration diameter of the first separation filter is larger than a filtration diameter of the second separation filter. The microbial liquid subjected to secondary filtration is introduced into the first separation filter for primary separation; the microbial liquid without passing through a first filter mesh is introduced into a first input end of a multi-channel single-cell sorter through a first output end of the first separation filter; the microbial liquid passing through the first filter mesh is introduced into the second separation filter through a second output end of the first separation filter for secondary separation; the microbial liquid without passing through a second filter mesh is introduced into a second input end of the multi-channel single-cell sorter through a first output end of the second separation filter; and the microbial liquid passing through the second filter mesh is introduced into a third input end of the multi-channel single-cell sorter through a second output end of the second separation filter to achieve multi-stage separation of different-size cells of the microbial liquid. After the microbial liquid is introduced into the multi-channel single-cell sorter, the state and type of individual cells entering each input end are observed through a viewing window on the multi-channel single-cell sorter to determine whether target microorganism cells have been obtained. If the target microorganism cells are not obtained, the microbial liquid subjected to primary filtration in the microorganism separator is renewed and the above steps are repeated until the target microorganism cells are obtained.

Preferably, the first filter includes a first filter frame and a first filtration membrane, and a surface of the first filter frame is covered with the first filtration membrane;
the second filter includes a second filter frame and a second filtration membrane, and a surface of the second filter frame is covered with the second filtration membrane; and
a filtration diameter of the second filtration membrane is smaller than a filtration diameter of the first filtration membrane.

The first filter is used to filter out large particulate impurities such as humus during the enrichment culture process of the microbial liquid, while the second filter is used to filter out sediments with a larger diameter than that of the microorganisms from the separated diluted microbial liquid.

Preferably, the first separation filter includes a first protective shell and a first filter mesh, and the first filter mesh is disposed inside the first protective shell;
the second separation filter includes a second protective shell and a second filter mesh, and the second filter mesh is disposed inside the second protective shell; and
a filtration diameter of the second filter mesh is smaller than a filtration diameter of the first filter mesh.

The first separation filter and the second separation filter are used to separate cells of different diameters. Since the filtration diameter of the second filter mesh is smaller than the filtration diameter of the first filter mesh, the microbial liquid subjected to secondary filtration is introduced into the first separation filter for primary separation; the microbial liquid without passing through the first filter mesh has a largest cell size and is introduced into the multi-channel single-cell sorter through the first output end of the first separation filter; the microbial liquid passing through the first filter mesh is introduced into the second separation filter for secondary separation through the second output end of the first separation filter; the microbial liquid without passing through the second filter mesh has an intermediate cell size and is introduced into the multi-channel single-cell sorter through the first output end of the second separation filter; the microbial liquid passing through the second filter mesh has a smallest cell size and is introduced into the multi-channel single-cell sorter through the second output end of the second separation filter.

Preferably, the microorganism separator includes a separation chamber, a diluent storage chamber, a suction filtration pump, a magnetic device, and magnetic beads;
the magnetic beads are disposed inside the separation chamber, and the magnetic device is disposed on a lower surface of the separation chamber and is corresponding to a position of the magnetic beads;
a water outlet end of the suction filtration pump is suspended, and a water inlet end of the suction filtration pump extends into an interior of the separation chamber; and a control end of the suction filtration pump is connected to a control end of the central control unit; and
a diluent is accommodated in the diluent storage chamber, and the diluent storage chamber is connected to an upper surface of the separation chamber via a pipeline.

After the microbial liquid subjected to primary filtration is introduced into the separation chamber of the microorganism separator, the diluent storage chamber is activated, and a diluent is introduced into the separation chamber to dilute the microbial liquid subjected to primary filtration according to a preset ratio, and the diluted microbial liquid is obtained. The diluent is mainly used to dilute the microbial liquid and protect cells from damage, and can also provide certain nutrients; the function of the magnetic device and magnetic beads is to drive the diluted microbial liquid inside the separation chamber to rotate, thereby separating a part where sediments and cells are bonded and obtaining the separated diluted microbial liquid. The function of the suction filtration pump is that, when the multi-channel single-cell sorter does not obtain target microorganism cells, the suction filtration pump is used to extract and discharge the microbial liquid subjected to primary filtration in the separation chamber, the microbial liquid subjected to primary filtration in the separation chamber is renewed, and the separation process is repeated again.

Preferably, the monitoring and cultivation unit includes a cultivation chamber, a support frame, a plurality of cultivation cups, a handle, an environmental indicator sensor, a first liquid injection pump, and a stabilization solution reservoir;
the environmental indicator sensor, the support frame, and the plurality of cultivation cups are disposed inside the cultivation chamber, and a data output end of the environmental indicator sensor is connected to a data input end of the central control unit;
the plurality of cultivation cups are evenly arranged on an upper surface of the support frame along a circumference; the handle is vertically disposed, with one end of the handle connected to the lower surface of the support frame and the other end of the handle extending out from a bottom surface of the cultivation chamber;
an upper surface of the cultivation chamber is provided with a first through hole and a second through hole, and positions of the first through hole and the second through hole correspond to any two of the cultivation cups; and
the first output end, second output end, and third output end of the multi-channel single-cell sorter are connected to the first through hole; and an output end of the stabilization solution reservoir is connected to one end of the first liquid injection pump, and the other end of the first liquid injection pump is connected to the second through hole.

Preferably, the environmental indicator sensor includes an environmental pressure sensor, an environmental temperature sensor, and a gas concentration detector.

The cultivation chamber is cylindrical, the support frame is circular, and the plurality of cultivation cups are evenly arranged on an upper surface of the support frame along a circumference. The handle is vertically connected to the lower surface of the support frame, and the upper surface of the cultivation chamber is provided with a first through hole and a second through hole that are corresponding to any two cultivation cups respectively. During use, the output end of the multi-channel single-cell sorter corresponding to the target microorganism cell is activated, and the target microorganism cell enters the cultivation cup through the first through hole for cultivation; the handle is rotated such that the next cultivation cup aligns with the first through hole; after a period of cultivation, the handle is rotated such that the cultivation cup containing the cultivated target microorganism cell aligns with the second through hole, the first liquid injection pump is controlled to activate to introduce a stabilization solution into the cultivation cup, thereby completing stabilization of the cultivated target microorganism cell and obtaining a stabilized sample.

Preferably, the microorganism enrichment unit includes a microorganism enrichment kettle, a magnetic stirrer, a vent valve, a liquid inlet valve, a pressure sensor, a temperature sensor, a water bath, and an intake valve;
a liquid outlet hole is provided in a side wall of the microorganism enrichment kettle, and the liquid outlet hole serves as a liquid outlet end of the microorganism enrichment unit and is connected to an input end of the first filter;
the microorganism enrichment kettle is disposed inside the water bath, and the magnetic stirrer is disposed on a lower surface of the microorganism enrichment kettle; control ends of the water bath and the magnetic stirrer are connected to a control end of the central control unit;
one end of the vent valve is connected to an upper surface of the microorganism enrichment kettle, and the other end of the vent valve is suspended; a control end of the vent valve is connected to a control end of the central control unit;
one end of the intake valve is connected to the upper surface of the microorganism enrichment kettle, and the other end of the intake valve serves as the gas inlet end of the microorganism enrichment unit and is connected to a gas outlet end of the gas injection unit; a control end of the intake valve is connected to a control end of the central control unit;
one end of the liquid inlet valve is connected to the side wall of the microorganism enrichment kettle, and the other end of the liquid inlet valve serves as the liquid inlet end of the microorganism enrichment unit and is connected to the liquid outlet end of the multi-sequence nutrient solution supply unit; a control end of the liquid inlet valve is connected to the control end of the central control unit; and
the temperature sensor and the pressure sensor are each disposed on the upper surface of the microorganism enrichment kettle; and data output ends of the temperature sensor and the pressure sensor are each connected to the data input end of the central control unit.

The upper surface of the microorganism enrichment kettle is detachable, facilitating sterilization and placement of substrates to be cultured; the lower surface of the microorganism enrichment kettle is provided with a magnetic stirrer to enhance mass transfer within the microorganism enrichment kettle and increase energy and nutrient utilization efficiency of the microbial liquid. The pressure sensor and the temperature sensor are used to monitor real-time changes in pressure and temperature inside the microorganism enrichment kettle, the vent valve is used to reduce the real-time pressure inside the microorganism enrichment kettle, and the water bath is used to maintain a constant temperature inside the microorganism enrichment kettle. The intake valve and the liquid inlet valve are respectively used to control the entry of the culture gas or inert gas and nutrient solution, ensuring that the microbial liquid is always in an optimal growth environment.

Preferably, the gas injection unit includes a booster pump, an air compressor, a gas storage tank, and a pressure regulating valve that are connected in sequence;
the other end of the pressure regulating valve serves as the gas outlet end of the gas injection unit and is connected to the other end of the intake valve; control ends of the booster pump, the air compressor, and the pressure regulating valve are connected to the control end of the central control unit; and
the culture gas or inert gas is accommodated in the gas storage tank.

Preferably, the multi-sequence nutrient solution supply unit includes a plurality of nutrient bottles, a plurality of liquid outlet valves, and a second liquid injection pump;
a liquid outlet of each nutrient bottle is correspondingly connected to one end of one liquid outlet valve; the other end of each liquid outlet valve is connected to one end of the second liquid injection pump; the other end of the second liquid injection pump serves as the liquid outlet end of the multi-sequence nutrient solution supply unit and is connected to the other end of the liquid inlet valve; control ends of each liquid outlet valve and the second liquid injection pump are connected to the control end of the central control unit; and
one nutrient solution is accommodated in each nutrient bottle.

The present invention further provides a continuous separation method for marine microorganisms under high-pressure conditions, and the method is applied to the above-mentioned continuous separation apparatus and includes:
S1: sterilizing a microorganism enrichment unit, a multi-stage separation unit, and a monitoring and cultivation unit; adding a substrate to be cultured into a microorganism enrichment kettle, adding culture gas or inert gas into a gas storage tank, and adding a nutrient solution into a nutrient bottle;
S2: controlling a real-time temperature of a water bath; controlling an intake valve to activated, adjusting a real-time pressure of the culture gas or inert gas output by a gas injection unit, and introducing the culture gas or inert gas into the microorganism enrichment kettle; controlling a liquid inlet valve to be activated, selecting the nutrient solution, controlling a corresponding liquid outlet valve and a second liquid injection pump to be activated, and introducing the nutrient solution into the microorganism enrichment kettle to form a microbial liquid;
S3: controlling a magnetic stirrer to agitate the microbial liquid; after a first preset time, controlling a liquid injection pump to pump the microbial liquid into a first filter to filter out large particulate impurities, obtaining the microbial liquid subjected to primary filtration, and introducing the microbial liquid into a separation chamber of a microorganism separator;
S4: controlling a diluent storage chamber to be activated, introducing a diluent into the separation chamber, diluting the microbial liquid subjected to primary filtration according to a preset ratio to obtain the diluted microbial liquid, controlling a magnetic device to be activated, and driving, by the magnetic beads, the diluted microbial liquid within the separation chamber to rotate, so as to obtain a separated diluted microbial liquid;
S5: introducing the separated diluted microbial liquid into a second filter to filter out sediments, so as to obtain the microbial liquid subjected to secondary filtration;
S6: introducing the microbial liquid subjected to secondary filtration into a first separation filter; introducing the microbial liquid without passing through a first filter mesh into a first input end of a multi-channel single-cell sorter through a first output end of a first separation filter; introducing the microbial liquid passing through the first filter mesh into a second filter through a second output end of the first separation filter; introducing the microbial liquid without passing through a second filter mesh into a second input end of the multi-channel single-cell sorter through a first output end of a second separation filter; and introducing the microbial liquid passing through the second filter mesh into a third input end of the multi-channel single-cell sorter through a second output end of the second separation filter;
S7: observing the state and type of individual cells entering each input end through a viewing window on the multi-channel single-cell sorter to determine whether target microbial cells are obtained; if target microbial cells are not obtianed, proceeding to step S8; otherwise, proceeding to step S9;
S8: controlling a suction filtration pump to be activated to extract and discharge the microbial liquid from the separation chamber, and repeating steps S3-S7;
S9: controlling an output end of the multi-channel single-cell sorter corresponding to the target microbial cell to be activated; rotating a handle such that a cultivation cup aligns with a first through hole, and allowing the target microbial cell to enter the cultivation cup for cultivation; after a second preset time, rotating the handle such that the cultivation cup aligns with a second through hole, and controlling a first liquid injection pump to be activated to introduce a stabilization solution into the cultivation cup, completing stabilization of the cultivated target microbial cell and obtaining a stabilized sample; and
S10: transferring the stabilized sample to an external multi-omics unit for cell characteristic detection.

Preferably, the external multi-omics unit includes an automatic DNA extraction instrument and a single-cell sequencer.

The automatic DNA extraction instrument and the single-cell sequencer can obtain genetic characteristics of individual microorganisms, thereby improving the detection efficiency and genome understanding of the separated individual microorganisms.

Compared with the prior art, the beneficial effects of the technical solutions of the present invention are as follows:

In the present invention, a microorganism enrichment unit is provided for microbial enrichment; a multi-sequence nutrient solution supply unit is used to supply a nutrient solution required for cultivation for the microorganism enrichment unit; a gas injection unit is used to supply culture gas or inert gas required for cultivation to the microorganism enrichment unit and a pressure within the microorganism enrichment unit enabled to be consistent with pressure of an in situ marine where the microorganisms is located, thereby guaranteeing the effectiveness of enrichment culture; after a period of enrichment culture, a microbial liquid is pumped into a multi-stage separation unit to achieve multi-stage separation of different-size cells of the microbial liquid, thereby obtaining target microbial cells; and finally, the target microbial cells are transferred to a monitoring and cultivation unit for cultivation and stabilization, and an external multi-omics unit is used to perform cell characteristic detection using an external multi-omics unit. The present invention enables continuous separation and characteristic detection of marine microorganisms of different sizes under in situ high-pressure marine environments. The present invention features simple operation, improved separation efficiency, accurate detection results, and good conformity to an actual environment.

### BRIEF DESCRIPTION OF DRAWINGS

FIG. 1 is a schematic structural diagram of a continuous separation apparatus for marine microorganisms under high-pressure conditions according to Embodiment 1.

FIG. 2 is a schematic structural diagram of a continuous separation apparatus for marine microorganisms under high-pressure conditions according to Embodiment 2.

FIG. 3 is a schematic structural diagram of a microorganism separator according to Embodiment 2.

FIG. 4 is a schematic diagram showing distribution of cultivation cups on a support frame according to Embodiment 2.

FIG. 5 is a flowchart of a continuous separation method for marine microorganisms under high-pressure conditions according to Embodiment 3.

### DETAILED DESCRIPTION OF EMBODIMENTS

The drawings are for exemplary illustration only and shall not be construed as limitations to this patent.

To better illustrate this embodiment, certain components in the drawings may be omitted, enlarged, or reduced, and do not represent the dimensions of the actual product.

For those skilled in the art, it is understandable that certain well-known structures and descriptions thereof in the drawings may be omitted.

The technical solutions of the present invention are further illustrated below in conjunction with the drawings and embodiments.

### Embodiment 1

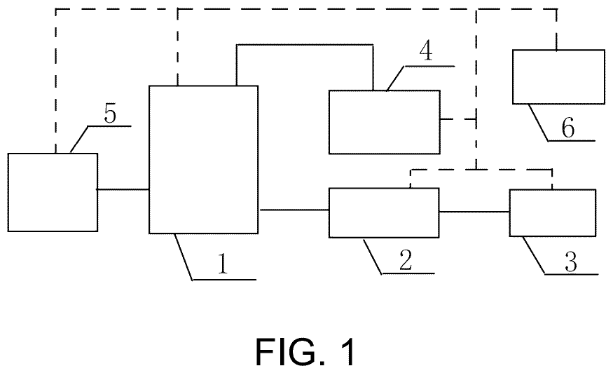

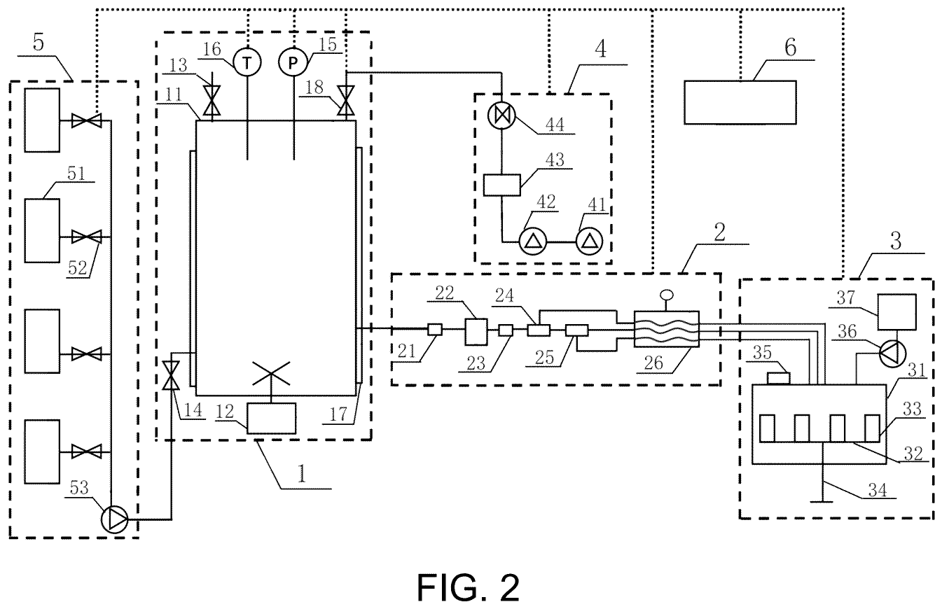

This embodiment discloses a continuous separation apparatus for marine microorganisms under high-pressure conditions. As shown in FIG. 1, the apparatus includes: a microorganism enrichment unit 1, a gas injection unit 4, a multi-sequence nutrient solution supply unit 5, a multi-stage separation unit 2, a monitoring and cultivation unit 3, and a central control unit 6;

a gas inlet end of the microorganism enrichment unit 1 is connected to a gas outlet end of the gas injection unit 4, and a liquid inlet end of the microorganism enrichment unit 1 is connected to a liquid outlet end of the multi-sequence nutrient solution supply unit 5;
an input end of the multi-stage separation unit 2 is connected to a liquid outlet end of the microorganism enrichment unit 1, and an output end of the multi-stage separation unit 2 is connected to an input end of the monitoring and cultivation unit 3; and
data output ends of the microorganism enrichment unit 1 and the monitoring and cultivation unit 3 are connected to a data input end of the central control unit 6; and control ends of the microorganism enrichment unit 1, the gas injection unit 4, the multi-sequence nutrient solution supply unit 5, the multi-stage separation unit 2, and the monitoring and cultivation unit 3 are connected to a control end of the central control unit 6.

In a specific implementation process, in this embodiment, the microorganism enrichment unit 1 is provided for microbial enrichment; the multi-sequence nutrient solution supply unit 5 is used to supply a nutrient solution required for microorganism cultivation for the microorganism enrichment unit 1; the gas injection unit 4 is used to supply culture gas or inert gas required for microorganism cultivation for the microorganism enrichment unit 1; and a pressure within the microorganism enrichment unit 1 is enabled to be consistent with pressure of an in situ marine where the microorganisms is located, thereby guaranteeing the effectiveness of enrichment culture; after a period of enrichment culture, a microbial liquid is pumped into the multi-stage separation unit 2 to achieve multi-stage separation of different-size cells of the microbial liquid, thereby obtaining target microbial cells; and finally, the target microbial cells are transferred to the monitoring and cultivation unit 3 for cultivation and stabilization, and an external multi-omics unit is used to perform cell characteristic detection.

### Embodiment 2

The present invention discloses a continuous separation apparatus for marine microorganisms under high-pressure conditions, and the apparatus includes: a microorganism enrichment unit 1, a gas injection unit 4, a multi-sequence nutrient solution supply unit 5, a multi-stage separation unit 2, a monitoring and cultivation unit 3, and a central control unit 6;
a gas inlet end of the microorganism enrichment unit 1 is connected to a gas outlet end of the gas injection unit 4, and a liquid inlet end of the microorganism enrichment unit 1 is connected to a liquid outlet end of the multi-sequence nutrient solution supply unit 5;
an input end of the multi-stage separation unit 2 is connected to a liquid outlet end of the microorganism enrichment unit 1, and an output end of the multi-stage separation unit 2 is connected to an input end of the monitoring and cultivation unit 3;
the microorganism enrichment unit 1 includes a microorganism enrichment kettle 11, a magnetic stirrer 12, a vent valve 13, a liquid inlet valve 14, a pressure sensor 15, a temperature sensor 16, a water bath 17, and an intake valve 18;
a liquid outlet hole is provided in a side wall of the microorganism enrichment kettle 11, and the liquid outlet hole serves as a liquid outlet end of the microorganism enrichment unit 1 and is connected to an input end of the first filter 21;
the microorganism enrichment kettle 11 is disposed inside the water bath 17, and the magnetic stirrer 12 is disposed on a lower surface of the microorganism enrichment kettle 11; control ends of the water bath 17 and the magnetic stirrer 12 are connected to a control end of the central control unit 6;
one end of the vent valve 13 is connected to an upper surface of the microorganism enrichment kettle 11, and the other end of the vent valve 13 is suspended; a control end of the vent valve 13 is connected to a control end of the central control unit 6;
one end of the intake valve 18 is connected to the upper surface of the microorganism enrichment kettle 11, and the other end of the intake valve 18 serves as the gas inlet end of the microorganism enrichment unit 1 and is connected to a gas outlet end of the gas injection unit 4; a control end of the intake valve 18 is connected to a control end of the central control unit 6;
one end of the liquid inlet valve 14 is connected to the side wall of the microorganism enrichment kettle 11, and the other end of the liquid inlet valve 14 serves as the liquid inlet end of the microorganism enrichment unit 1 and is connected to the liquid outlet end of the multi-sequence nutrient solution supply unit 5; a control end of the liquid inlet valve 14 is connected to the control end of the central control unit 6; and
the temperature sensor 16 and the pressure sensor 15 are each disposed on the upper surface of the microorganism enrichment kettle 11; and data output ends of the temperature sensor 16 and the pressure sensor 15 are each connected to the data input end of the central control unit 6.

The upper surface of the microorganism enrichment kettle 11 is detachable, facilitating sterilization and placement of substrates to be cultured; the lower surface thereof is provided with a magnetic stirrer 12 to enhance mass transfer within the microorganism enrichment kettle 11 and increase energy and nutrient utilization efficiency of the microbial liquid. The pressure sensor 15 and the temperature sensor 16 are used to monitor real-time changes in pressure and temperature inside the microorganism enrichment kettle 11, the vent valve 13 is used to reduce the real-time pressure inside the microorganism enrichment kettle 11, and the water bath 17 is used to maintain a constant temperature inside the microorganism enrichment kettle 11. The intake valve 18 and the liquid inlet valve 14 are respectively used to control the entry of the culture gas or inert gas and nutrient solution, ensuring that the microbial liquid is always in an optimal growth environment.

The gas injection unit 4 includes a booster pump 41, an air compressor 42, a gas storage tank 43, and a pressure regulating valve 44 that are connected in sequence;
the other end of the pressure regulating valve 44 serves as the gas outlet end of the gas injection unit 4 and is connected to the other end of the intake valve 18; control ends of the booster pump 41, the air compressor 42, and the pressure regulating valve 44 are connected to the control end of the central control unit 6; and
culture gas or inert gas is accommodated in the gas storage tank 43.

The multi-sequence nutrient solution supply unit 5 includes a plurality of nutrient bottles 51, a plurality of liquid outlet valves 52, and a second liquid injection pump 53;
a liquid outlet of each nutrient bottle 51 is correspondingly connected to one end of one liquid outlet valve 52; the other end of each liquid outlet valve 52 is connected to one end of the second liquid injection pump 53; the other end of the second liquid injection pump 53 serves as the liquid outlet end of the multi-sequence nutrient solution supply unit 5 and is connected to the other end of the liquid inlet valve 14; control ends of each liquid outlet valve 52 and control ends the second liquid injection pump 53 are connected to the control end of the central control unit 6; and
one nutrient solution is accommodated in each nutrient bottle 51.

The multi-stage separation unit 2 includes a first filter 21, a microorganism separator 22, a second filter 23, a first separation filter 24, a second separation filter 25, and a multi-channel single-cell sorter 26;
the first filter 21, the microorganism separator 22, the second filter 23, and the first separation filter 24 are connected in sequence; a first output end of the first separation filter 24 is connected to a first input end of the multi-channel single-cell sorter 26, a second output end of the first separation filter 24 is connected to an input end of the second separation filter 25, a first output end of the second separation filter 25 is connected to a second input end of the multi-channel single-cell sorter 26, and a second output end of the second separation filter 25 is connected to a third input end of the multi-channel single-cell sorter 26;
an input end of the first filter 21 serves as the input end of the multi-stage separation unit 2 and is connected to the liquid outlet end of the microorganism enrichment unit 1; the first output end, second output end, and third output end of the multi-channel single-cell sorter 26 all serve as the output end of the multi-stage separation unit 2 and are connected to the input end of the monitoring and cultivation unit 3;
the first filter 21 includes a first filter frame and a first filtration membrane, and a surface of the first filter frame is covered with the first filtration membrane;
the second filter 23 includes a second filter frame and a second filtration membrane, and a surface of the second filter frame is covered with the second filtration membrane; and
a filtration diameter of the second filtration membrane is smaller than a filtration diameter of the first filtration membrane;
the first separation filter 24 includes a first protective shell and a first filter mesh, and the first filter mesh is disposed inside the first protective shell;
the second separation filter 25 includes a second protective shell and a second filter mesh, and the second filter mesh is disposed inside the second protective shell; and
a filtration diameter of the second filter mesh is smaller than a filtration diameter of the first filter mesh.

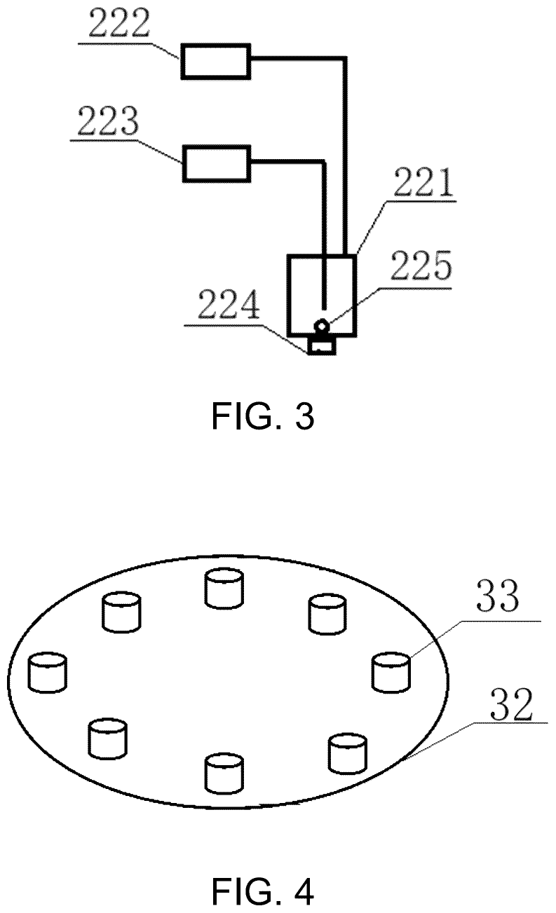

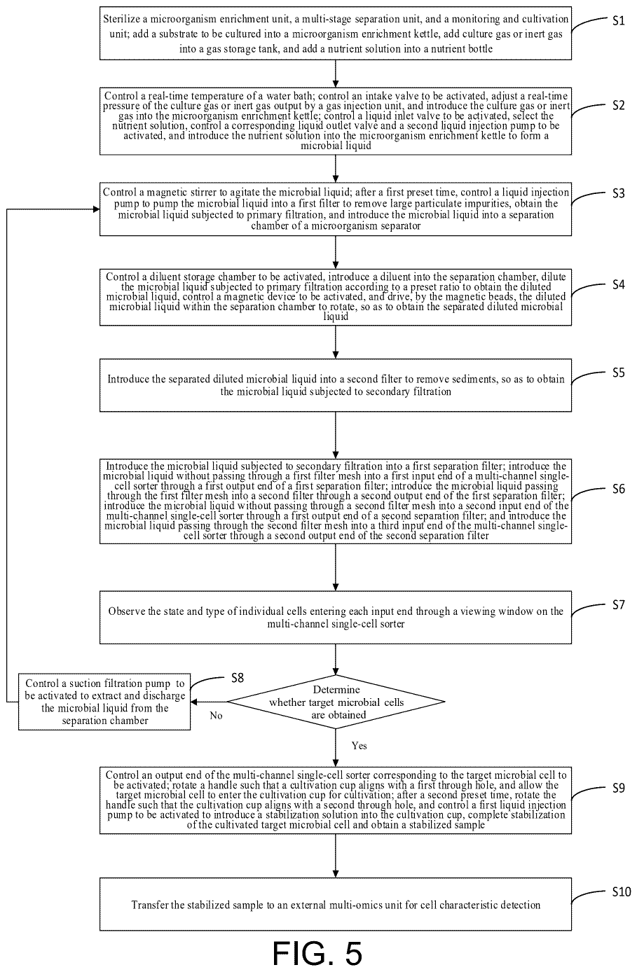

As shown in FIG. 3, the microorganism separator 22 includes a separation chamber 221, a diluent storage chamber 222, a suction filtration pump 223, a magnetic device 224, and magnetic beads 225;
the magnetic beads 225 are disposed inside the separation chamber 221, and the magnetic device 224 is disposed on a lower surface of the separation chamber 221, and is corresponding to a position of the magnetic beads 225;
a water outlet end of the suction filtration pump 223 is suspended, and a water inlet end of the suction filtration pump 223 extends into an interior of the separation chamber 221; and a control end of the suction filtration pump 223 is connected to a control end of the central control unit 6; and
a diluent is accommodated in the diluent storage chamber 222, and the diluent storage chamber 222 is connected to an upper surface of the separation chamber 221 via a pipeline.

The microbial liquid is pumped into the first filter 21 to filter out large particulate impurities such as humus, the microbial liquid subjected to primary filtration is obtained and introduced into a microorganism separator 22 for dilution and separation. After the microbial liquid subjected to primary filtration is introduced into the separation chamber 221 of the microorganism separator 22, the diluent storage chamber 222 is activated, a diluent is introduced into the separation chamber 221 to dilute the microbial liquid subjected to primary filtration according to a preset ratio, and the diluted microbial liquid is obtained. The diluent is mainly used to dilute the suspension and protect cells from damage, and can provide certain nutrients; the function of the magnetic device 224 and magnetic beads 225 is to drive the diluted microbial liquid inside the separation chamber 221 to rotate, thereby separating a part where sediments and cells are bonded and obtaining the separated diluted microbial liquid; the separated diluted microbial liquid is introduced into a second filter 23 to filter out sediments that are from the separated diluted microbial liquid and have a larger diameter than the microorganisms, so as to obtain the microbial liquid subjected to secondary filtration. A filtration diameter of the first separation filter 24 is larger than a filtration diameter of the second separation filter 25. The microbial liquid subjected to secondary filtration is introduced into the first separation filter 24 for primary separation; the microbial liquid without passing through a first filter mesh is introduced into a first input end of a multi-channel single-cell sorter 26 through a first output end of the first separation filter 24; the microbial liquid passing through the first filter mesh is introduced into the second filter 23 through a second output end of the first separation filter 24 for secondary separation; the microbial liquid without passing through a second filter mesh is introduced into a second input end of the multi-channel single-cell sorter 26 through a first output end of the second separation filter 25; and the microbial liquid passing through the second filter mesh is introduced into a third input end of the multi-channel single-cell sorter 26 through a second output end of the second separation filter 25 to achieve multi-stage separation of different-size cells of the microbial liquid. The first separation filter 24 and the second separation filter 25 are used to separate cells of different diameters. Since the filtration diameter of the second filter mesh 252 is smaller than the filtration diameter of the first filter mesh 242, the microbial liquid subjected to secondary filtration is introduced into the first separation filter 24 for primary separation; the microbial liquid without passing through the first filter mesh 242 has a largest cell size and is introduced into the multi-channel single-cell sorter 26 through the first output end of the first separation filter 24; the microbial liquid passing through the first filter mesh is introduced into the second separation filter 25 for secondary separation through the second output end of the first separation filter 24; the microbial liquid without passing through the second filter mesh 252 has an intermediate cell size and is introduced into the multi-channel single-cell sorter 26 through the first output end of the second separation filter 25; and the microbial liquid passing through the second filter mesh 252 has a smallest cell size and is introduced into the multi-channel single-cell sorter 26 through the second output end of the second separation filter 25. After the microbial liquid is introduced into the multi-channel single-cell sorter 26, the state and type of individual cells entering each input end are observed through a viewing window on the multi-channel single-cell sorter 26 to determine whether target microorganism cells are obtained. If the target microorganism cells are not obtained, the suction filtration pump 223 is used to extract and discharge the microbial liquid subjected to primary filtration in the separation chamber 221, the microbial liquid subjected to primary filtration in the microorganism separator 22 is renewed and the above steps are repeated until the target microorganism cells are obtained.

The monitoring and cultivation unit 3 includes a cultivation chamber 31, a support frame 32, a plurality of cultivation cups 33, a handle 34, an environmental indicator sensor 35, a first liquid injection pump 36, and a stabilization solution reservoir 37; and
the environmental indicator sensor 35, the support frame 32, and the plurality of cultivation cups 33 are disposed inside the cultivation chamber 31; a data output end of the environmental indicator sensor 35 is connected to a data input end of the central control unit 6.

As shown in FIG. 4, the plurality of cultivation cups 33 are evenly arranged on an upper surface of the support frame 32 along a circumference; the handle 34 is vertically disposed, with one end of the handle 34 connected to the lower surface of the support frame 32 and the other end of the handle 34 extending out from a bottom surface of the cultivation chamber 31;
an upper surface of the cultivation chamber 31 is provided with a first through hole and a second through hole, and positions of the first through hole and the second through hole correspond to any two of the cultivation cups 33;
the first output end, second output end, and third output end of the multi-channel single-cell sorter 26 are connected to the first through hole; and an output end of the stabilization solution reservoir 37 is connected to one end of the first liquid injection pump 36, and the other end of the first liquid injection pump 36 is connected to the second through hole; and
the environmental indicator sensor 35 includes an environmental pressure sensor, an environmental temperature sensor, and a gas concentration detector.

The cultivation chamber 31 is cylindrical, the support frame 32 is circular, and the plurality of cultivation cups 33 are evenly arranged on an upper surface of the support frame 32 along a circumference. The handle 34 is vertically connected to the lower surface of the support frame 32, and the upper surface of the cultivation chamber 31 is provided with a first through hole and a second through hole that are respectively corresponding to any two cultivation cups 33. During use, the output end of the multi-channel single-cell sorter 26 corresponding to the target microorganism cell is activated, and the target microorganism cell enters the cultivation cup 33 through the first through hole for cultivation; the handle 34 is rotated such that the next cultivation cup 33 aligns with the first through hole; after a period of cultivation, the handle 34 is rotated such that the cultivation cup 33 containing the cultivated target microorganism cell aligns with the second through hole, the first liquid injection pump 36 is controlled to be activated to introduce a stabilization solution into the cultivation cup 33, thereby completing stabilization of the cultivated target microorganism cell and obtaining a stabilized sample.

This embodiment enables characteristic detection after continuous separation different-size marine microorganisms under in situ high-pressure marine environments. The present invention features simple operation, improved separation efficiency, accurate detection results, and good conformity to an actual environment.

### Embodiment 3

This embodiment provides a continuous separation method for marine microorganisms under high-pressure conditions, and the method is applied to the continuous separation apparatus described in Embodiment 1 or 2, as shown in FIG. 3, and includes:
S1: sterilizing a microorganism enrichment unit, a multi-stage separation unit, and a monitoring and cultivation unit; adding a substrate to be cultured into a microorganism enrichment kettle, adding culture gas or inert gas into a gas storage tank, and adding a nutrient solution into a nutrient bottle;
S2: controlling a real-time temperature of a water bath; controlling an intake valve to be activated, adjusting a real-time pressure of the culture gas or inert gas output by a gas injection unit, and introducing the culture gas or inert gas into the microorganism enrichment kettle; controlling a liquid inlet valve to be activated, selecting the nutrient solution, controlling a corresponding liquid outlet valve and a second liquid injection pump to be activated, and introducing the nutrient solution into the microorganism enrichment kettle to form a microbial liquid;
S3: controlling a magnetic stirrer to agitate the microbial liquid; after a first preset time, controlling a liquid injection pump to pump the microbial liquid into a first filter to filter out large particulate impurities, obtaining the microbial liquid subjected to primary filtration, and introducing the microbial liquid into a separation chamber of a microorganism separator;
S4: controlling a diluent storage chamber to be activated, introducing a diluent into the separation chamber, diluting the microbial liquid subjected to primary filtration according to a preset ratio to obtain the diluted microbial liquid, controlling a magnetic device to be activated, and driving, by magnetic beads, the diluted microbial liquid within the separation chamber to rotate, so as to obtain a separated diluted microbial liquid;
S5: introducing the separated diluted microbial liquid into a second filter to filter out sediments, so as to obtain the microbial liquid subjected to secondary filtration;
S6: introducing the microbial liquid subjected to secondary filtration into a first separation filter; introducing the microbial liquid without passing through a first filter mesh into a first input end of a multi-channel single-cell sorter through a first output end of a first separation filter; introducing the microbial liquid passing through the first filter mesh into a second filter through a second output end of the first separation filter; introducing the microbial liquid without passing through a second filter mesh into a second input end of the multi-channel single-cell sorter through a first output end of a second separation filter; and introducing the microbial liquid passing through the second filter mesh into a third input end of the multi-channel single-cell sorter through a second output end of the second separation filter;
S7: observing the state and type of individual cells entering each input end through a viewing window on the multi-channel single-cell sorter to determine whether target microbial cells are obtained; if the target microbial cells are not obtained, proceeding to step S8; otherwise, proceeding to step S9;
S8: controlling a suction filtration pump to be activated to extract and discharge the microbial liquid from the separation chamber, and repeating steps S3-S7;
S9: controlling an output end of the multi-channel single-cell sorter corresponding to the target microbial cell to be activated; rotating a handle such that a cultivation cup aligns with a first through hole, and allowing the target microbial cell to enter the cultivation cup for cultivation; after a second preset time, rotating the handle such that the cultivation cup aligns with a second through hole, and controlling a first liquid injection pump to be activated to introduce a stabilization solution into the cultivation cup, completing stabilization of the cultivated target microbial cell and obtaining a stabilized sample; and
S10: transferring the stabilized sample to an external multi-omics unit for cell characteristic detection.

The external multi-omics unit includes an automatic DNA extraction instrument and a single-cell sequencer.

The automatic DNA extraction instrument and the single-cell sequencer can obtain genetic characteristics of individual microorganisms, thereby improving the detection efficiency and genome understanding of the separated individual microorganisms.

Identical or similar reference numerals correspond to identical or similar components.

Terms describing positional relationships in the drawings are for exemplary illustration only and shall not be construed as limitations to this patent application.

Obviously, the above embodiments of the present invention are merely examples to clearly illustrate the present invention and are not intended to limit the implementations of the present invention. For those of ordinary skill in the art, other variations or modifications in different forms can be made based on the above description. It is neither necessary nor possible to exhaustively list all implementations herein. Any modifications, equivalent substitutions, improvements, and the like made within the spirit and principles of the present invention shall be included within the scope of protection of the claims of the present invention.

1. A continuous separation apparatus for marine microorganisms under high-pressure conditions, comprising: a microorganism enrichment unit, a multi-stage separation unit, a monitoring and cultivation unit, a gas injection unit, a multi-sequence nutrient solution supply unit, and a central control unit;
a gas inlet end of the microorganism enrichment unit is connected to a gas outlet end of the gas injection unit, and a liquid inlet end of the microorganism enrichment unit is connected to a liquid outlet end of the multi-sequence nutrient solution supply unit;
an input end of the multi-stage separation unit is connected to a liquid outlet end of the microorganism enrichment unit, and an output end of the multi-stage separation unit is connected to an input end of the monitoring and cultivation unit;
data output ends of the microorganism enrichment unit and the monitoring and cultivation unit are connected to a data input end of the central control unit; and control ends of the microorganism enrichment unit, the gas injection unit, the multi-sequence nutrient solution supply unit, the multi-stage separation unit, and the monitoring and cultivation unit are connected to a control end of the central control unit;
the multi-stage separation unit comprises a first filter, a microorganism separator, a second filter, a first separation filter, a second separation filter, and a multi-channel single-cell sorter;
the first filter, the microorganism separator, the second filter, and the first separation filter are connected in sequence; a first output end of the first separation filter is connected to a first input end of the multi-channel single-cell sorter, a second output end of the first separation filter is connected to an input end of the second separation filter, a first output end of the second separation filter is connected to a second input end of the multi-channel single-cell sorter, and a second output end of the second separation filter is connected to a third input end of the multi-channel single-cell sorter;
an input end of the first filter serves as the input end of the multi-stage separation unit and is connected to the liquid outlet end of the microorganism enrichment unit; the first output end, second output end, and third output end of the multi-channel single-cell sorter all serve as the output end of the multi-stage separation unit and are connected to the input end of the monitoring and cultivation unit;
the microorganism separator comprises a separation chamber, a diluent storage chamber, a suction filtration pump, a magnetic device, and magnetic beads;
the magnetic beads are disposed inside the separation chamber, and the magnetic device is disposed on a lower surface of the separation chamber and is corresponding to a position of the magnetic beads;
a water outlet end of the suction filtration pump is suspended, and a water inlet end of the suction filtration pump extends into an interior of the separation chamber; and a control end of the suction filtration pump is connected to a control end of the central control unit; and
a diluent is accommodated in the diluent storage chamber, and the diluent storage chamber is connected to an upper surface of the separation chamber via a pipeline.

2. The continuous separation apparatus for marine microorganisms under high-pressure conditions according to claim 1, wherein the first filter comprises a first filter frame and a first filtration membrane, and a surface of the first filter frame is covered with the first filtration membrane;
the second filter comprises a second filter frame and a second filtration membrane, and a surface of the second filter frame is covered with the second filtration membrane; and
a filtration diameter of the second filtration membrane is smaller than a filtration diameter of the first filtration membrane.

3. The continuous separation apparatus for marine microorganisms under high-pressure conditions according to claim 1, wherein the first separation filter comprises a first protective shell and a first filter mesh, and the first filter mesh is disposed inside the first protective shell;
the second separation filter comprises a second protective shell and a second filter mesh, and the second filter mesh is disposed inside the second protective shell; and
a filtration diameter of the second filter mesh is smaller than a filtration diameter of the first filter mesh.

4. The continuous separation apparatus for marine microorganisms under high-pressure conditions according to claim 1, wherein the monitoring and cultivation unit comprises a cultivation chamber, a support frame, a plurality of cultivation cups, a handle, an environmental indicator sensor, a first liquid injection pump, and a stabilization solution reservoir;
the environmental indicator sensor, the support frame, and the plurality of cultivation cups are disposed inside the cultivation chamber; a data output end of the environmental indicator sensor is connected to a data input end of the central control unit;
the plurality of cultivation cups are evenly arranged on an upper surface of the support frame along a circumference; the handle is vertically disposed, with one end of the handle connected to a lower surface of the support frame and the other end of the handle extending out from a bottom surface of the cultivation chamber;
an upper surface of the cultivation chamber is provided with a first through hole and a second through hole, and positions of the first through hole and the second through hole correspond to any two of the cultivation cups; and
the first output end, second output end, and third output end of the multi-channel single-cell sorter are connected to the first through hole; and an output end of the stabilization solution reservoir is connected to one end of the first liquid injection pump, and the other end of the first liquid injection pump is connected to the second through hole.

5. The continuous separation apparatus for marine microorganisms under high-pressure conditions according to claim 4, wherein the microorganism enrichment unit comprises a microorganism enrichment kettle, a magnetic stirrer, a vent valve, a liquid inlet valve, a pressure sensor, a temperature sensor, a water bath, and an intake valve;
a liquid outlet hole is provided in a side wall of the microorganism enrichment kettle, and the liquid outlet hole serves as a liquid outlet end of the microorganism enrichment unit and is connected to an input end of the first filter;
the microorganism enrichment kettle is disposed inside the water bath, and the magnetic stirrer is disposed on a lower surface of the microorganism enrichment kettle; control ends of the water bath and the magnetic stirrer are connected to a control end of the central control unit;
one end of the vent valve is connected to an upper surface of the microorganism enrichment kettle, and the other end of the vent valve is suspended; a control end of the vent valve is connected to a control end of the central control unit;
one end of the intake valve is connected to the upper surface of the microorganism enrichment kettle, and the other end of the intake valve serves as the gas inlet end of the microorganism enrichment unit and is connected to a gas outlet end of the gas injection unit; a control end of the intake valve is connected to a control end of the central control unit;
one end of the liquid inlet valve is connected to the side wall of the microorganism enrichment kettle, and the other end of the liquid inlet valve serves as the liquid inlet end of the microorganism enrichment unit and is connected to the liquid outlet end of the multi-sequence nutrient solution supply unit; a control end of the liquid inlet valve is connected to the control end of the central control unit; and
the temperature sensor and the pressure sensor are each disposed on the upper surface of the microorganism enrichment kettle; and data output ends of the temperature sensor and the pressure sensor are each connected to the data input end of the central control unit.

6. The continuous separation apparatus for marine microorganisms under high-pressure conditions according to claim 4, wherein the gas injection unit comprises a booster pump, an air compressor, a gas storage tank, and a pressure regulating valve that are connected in sequence;
the other end of the pressure regulating valve serves as the gas outlet end of the gas injection unit and is connected to the other end of the intake valve; control ends of the booster pump, the air compressor, and the pressure regulating valve are connected to the control end of the central control unit; and
culture gas or inert gas is accommodated in the gas storage tank.

7. The continuous separation apparatus for marine microorganisms under high-pressure conditions according to claim 4, wherein the multi-sequence nutrient solution supply unit comprises a plurality of nutrient bottles, a plurality of liquid outlet valves, and a second liquid injection pump;
a liquid outlet of each nutrient bottle is correspondingly connected to one end of one liquid outlet valve; the other end of each liquid outlet valve is connected to one end of the second liquid injection pump; the other end of the second liquid injection pump serves as the liquid outlet end of the multi-sequence nutrient solution supply unit and is connected to the other end of the liquid inlet valve; control ends of each liquid outlet valve and control ends of the second liquid injection pump are connected to the control end of the central control unit; and
one nutrient solution is accommodated in each nutrient bottle.

8. The continuous separation apparatus for marine microorganisms under high-pressure conditions according to claim 2, wherein the monitoring and cultivation unit comprises a cultivation chamber, a support frame, a plurality of cultivation cups, a handle, an environmental indicator sensor, a first liquid injection pump, and a stabilization solution reservoir;
the environmental indicator sensor, the support frame, and the plurality of cultivation cups are disposed inside the cultivation chamber; a data output end of the environmental indicator sensor is connected to a data input end of the central control unit;
the plurality of cultivation cups are evenly arranged on an upper surface of the support frame along a circumference; the handle is vertically disposed, with one end of the handle connected to a lower surface of the support frame and the other end of the handle extending out from a bottom surface of the cultivation chamber;
an upper surface of the cultivation chamber is provided with a first through hole and a second through hole, and positions of the first through hole and the second through hole correspond to any two of the cultivation cups; and
the first output end, second output end, and third output end of the multi-channel single-cell sorter are connected to the first through hole; and an output end of the stabilization solution reservoir is connected to one end of the first liquid injection pump, and the other end of the first liquid injection pump is connected to the second through hole.

9. The continuous separation apparatus for marine microorganisms under high-pressure conditions according to claim 8, wherein the microorganism enrichment unit comprises a microorganism enrichment kettle, a magnetic stirrer, a vent valve, a liquid inlet valve, a pressure sensor, a temperature sensor, a water bath, and an intake valve;
a liquid outlet hole is provided in a side wall of the microorganism enrichment kettle, and the liquid outlet hole serves as a liquid outlet end of the microorganism enrichment unit and is connected to an input end of the first filter;
the microorganism enrichment kettle is disposed inside the water bath, and the magnetic stirrer is disposed on a lower surface of the microorganism enrichment kettle; control ends of the water bath and the magnetic stirrer are connected to a control end of the central control unit;
one end of the vent valve is connected to an upper surface of the microorganism enrichment kettle, and the other end of the vent valve is suspended; a control end of the vent valve is connected to a control end of the central control unit;
one end of the intake valve is connected to the upper surface of the microorganism enrichment kettle, and the other end of the intake valve serves as the gas inlet end of the microorganism enrichment unit and is connected to a gas outlet end of the gas injection unit; a control end of the intake valve is connected to a control end of the central control unit;
one end of the liquid inlet valve is connected to the side wall of the microorganism enrichment kettle, and the other end of the liquid inlet valve serves as the liquid inlet end of the microorganism enrichment unit and is connected to the liquid outlet end of the multi-sequence nutrient solution supply unit; a control end of the liquid inlet valve is connected to the control end of the central control unit; and
the temperature sensor and the pressure sensor are each disposed on the upper surface of the microorganism enrichment kettle; and data output ends of the temperature sensor and the pressure sensor are each connected to the data input end of the central control unit.

10. The continuous separation apparatus for marine microorganisms under high-pressure conditions according to claim 8, wherein the gas injection unit comprises a booster pump, an air compressor, a gas storage tank, and a pressure regulating valve that are connected in sequence;
the other end of the pressure regulating valve serves as the gas outlet end of the gas injection unit and is connected to the other end of the intake valve; control ends of the booster pump, the air compressor, and the pressure regulating valve are connected to the control end of the central control unit; and
culture gas or inert gas is accommodated in the gas storage tank.

11. The continuous separation apparatus for marine microorganisms under high-pressure conditions according to claim 8, wherein the multi-sequence nutrient solution supply unit comprises a plurality of nutrient bottles, a plurality of liquid outlet valves, and a second liquid injection pump;
a liquid outlet of each nutrient bottle is correspondingly connected to one end of one liquid outlet valve; the other end of each liquid outlet valve is connected to one end of the second liquid injection pump; the other end of the second liquid injection pump serves as the liquid outlet end of the multi-sequence nutrient solution supply unit and is connected to the other end of the liquid inlet valve; control ends of each liquid outlet valve and control ends of the second liquid injection pump are connected to the control end of the central control unit; and
one nutrient solution is accommodated in each nutrient bottle.

12. The continuous separation apparatus for marine microorganisms under high-pressure conditions according to claim 3, wherein the monitoring and cultivation unit comprises a cultivation chamber, a support frame, a plurality of cultivation cups, a handle, an environmental indicator sensor, a first liquid injection pump, and a stabilization solution reservoir;
the environmental indicator sensor, the support frame, and the plurality of cultivation cups are disposed inside the cultivation chamber; a data output end of the environmental indicator sensor is connected to a data input end of the central control unit;
the plurality of cultivation cups are evenly arranged on an upper surface of the support frame along a circumference; the handle is vertically disposed, with one end of the handle connected to a lower surface of the support frame and the other end of the handle extending out from a bottom surface of the cultivation chamber;
an upper surface of the cultivation chamber is provided with a first through hole and a second through hole, and positions of the first through hole and the second through hole correspond to any two of the cultivation cups; and
the first output end, second output end, and third output end of the multi-channel single-cell sorter are connected to the first through hole; and an output end of the stabilization solution reservoir is connected to one end of the first liquid injection pump, and the other end of the first liquid injection pump is connected to the second through hole.

13. The continuous separation apparatus for marine microorganisms under high-pressure conditions according to claim 12, wherein the microorganism enrichment unit comprises a microorganism enrichment kettle, a magnetic stirrer, a vent valve, a liquid inlet valve, a pressure sensor, a temperature sensor, a water bath, and an intake valve;
a liquid outlet hole is provided in a side wall of the microorganism enrichment kettle, and the liquid outlet hole serves as a liquid outlet end of the microorganism enrichment unit and is connected to an input end of the first filter;
the microorganism enrichment kettle is disposed inside the water bath, and the magnetic stirrer is disposed on a lower surface of the microorganism enrichment kettle; control ends of the water bath and the magnetic stirrer are connected to a control end of the central control unit;
one end of the vent valve is connected to an upper surface of the microorganism enrichment kettle, and the other end of the vent valve is suspended; a control end of the vent valve is connected to a control end of the central control unit;
one end of the intake valve is connected to the upper surface of the microorganism enrichment kettle, and the other end of the intake valve serves as the gas inlet end of the microorganism enrichment unit and is connected to a gas outlet end of the gas injection unit; a control end of the intake valve is connected to a control end of the central control unit;
one end of the liquid inlet valve is connected to the side wall of the microorganism enrichment kettle, and the other end of the liquid inlet valve serves as the liquid inlet end of the microorganism enrichment unit and is connected to the liquid outlet end of the multi-sequence nutrient solution supply unit; a control end of the liquid inlet valve is connected to the control end of the central control unit; and
the temperature sensor and the pressure sensor are each disposed on the upper surface of the microorganism enrichment kettle; and data output ends of the temperature sensor and the pressure sensor are each connected to the data input end of the central control unit.

14. The continuous separation apparatus for marine microorganisms under high-pressure conditions according to claim 12, wherein the gas injection unit comprises a booster pump, an air compressor, a gas storage tank, and a pressure regulating valve that are connected in sequence;
the other end of the pressure regulating valve serves as the gas outlet end of the gas injection unit and is connected to the other end of the intake valve; control ends of the booster pump, the air compressor, and the pressure regulating valve are connected to the control end of the central control unit; and
culture gas or inert gas is accommodated in the gas storage tank.

15. The continuous separation apparatus for marine microorganisms under high-pressure conditions according to claim 12, wherein the multi-sequence nutrient solution supply unit comprises a plurality of nutrient bottles, a plurality of liquid outlet valves, and a second liquid injection pump;
a liquid outlet of each nutrient bottle is correspondingly connected to one end of one liquid outlet valve; the other end of each liquid outlet valve is connected to one end of the second liquid injection pump; the other end of the second liquid injection pump serves as the liquid outlet end of the multi-sequence nutrient solution supply unit and is connected to the other end of the liquid inlet valve; control ends of each liquid outlet valve and control ends of the second liquid injection pump are connected to the control end of the central control unit; and
one nutrient solution is accommodated in each nutrient bottle.

16. A continuous separation method for marine microorganisms under high-pressure conditions, applied to the continuous separation apparatus according to claim 1, comprising:
S1: sterilizing a microorganism enrichment unit, a multi-stage separation unit, and a monitoring and cultivation unit; adding a substrate to be cultured into a microorganism enrichment kettle, adding culture gas or inert gas into a gas storage tank, and adding a nutrient solution into a nutrient bottle;
S2: controlling a real-time temperature of a water bath; controlling an intake valve to be activated, adjusting a real-time pressure of the culture gas or inert gas output by a gas injection unit, and introducing the culture gas or inert gas into the microorganism enrichment kettle; controlling a liquid inlet valve to be activated, selecting the nutrient solution, controlling a corresponding liquid outlet valve and a second liquid injection pump to be activated, and introducing the nutrient solution into the microorganism enrichment kettle to form a microbial liquid;
S3: controlling a magnetic stirrer to agitate the microbial liquid; after a first preset time, controlling a liquid injection pump to pump the microbial liquid into a first filter to filter out large particulate impurities, obtaining the microbial liquid subjected to primary filtration, and introducing the microbial liquid into a separation chamber of a microorganism separator;
S4: controlling a diluent storage chamber to be activated, introducing a diluent into the separation chamber, diluting the microbial liquid subjected to primary filtration according to a preset ratio to obtain the diluted microbial liquid, controlling a magnetic device to be activated, and driving, by magnetic beads, the diluted microbial liquid within the separation chamber to rotate, so as to obtain a separated diluted microbial liquid;
S5: introducing the separated diluted microbial liquid into a second filter to filter out sediments, so as to obtain the microbial liquid subjected to secondary filtration;
S6: introducing the microbial liquid subjected to secondary filtration into a first separation filter; introducing the microbial liquid without passing through a first filter mesh into a first input end of a multi-channel single-cell sorter through a first output end of a first separation filter; introducing the microbial liquid passing through the first filter mesh into a second filter through a second output end of the first separation filter; introducing the microbial liquid without passing through a second filter mesh into a second input end of the multi-channel single-cell sorter through a first output end of a second separation filter; and introducing the microbial liquid passing through the second filter mesh into a third input end of the multi-channel single-cell sorter through a second output end of the second separation filter;
S7: observing the state and type of individual cells entering each input end through a viewing window on the multi-channel single-cell sorter to determine whether target microbial cells have been obtained; if microbial cells have not been obtained, proceeding to step S8; otherwise, proceeding to step S9;
S8: controlling a suction filtration pump to be activated to extract and discharge the microbial liquid from the separation chamber, and repeating steps S3-S7;
S9: controlling an output end of the multi-channel single-cell sorter corresponding to the target microbial cell to be activated; rotating a handle such that a cultivation cup aligns with a first through hole, and allowing the target microbial cell to enter the cultivation cup for cultivation; after a second preset time, rotating the handle such that the cultivation cup aligns with a second through hole, and controlling a first liquid injection pump to be activated to introduce a stabilization solution into the cultivation cup, completing stabilization of the cultivated target microbial cell and obtaining a stabilized sample; and
S10: transferring the stabilized sample to an external multi-omics unit for cell characteristic detection.
